(19) United States
(12) Patent Application Publication
(10) Pub. No.: US 2025/0257331 A1
(43) Pub. Date: Aug. 14, 2025

(54) FLAVONOID GLYCOSIDE GLYCOSYLTRANSFERASE LbUGT71BX1 IN LAPORTEA BULBIFERA AS WELL AS THE CODING GENE AND THE USE THEREOF

(71) Applicant: INSTITUTE OF CHINESE MATERIA MEDICA CHINA ACADEMY OF CHINESE MEDICAL SCIENCES, Beijing (CN)

(72) Inventors: Lan Wu, Beijing (CN); Li Xiang, Beijing (CN); Ranran Gao, Beijing (CN); Shilin Chen, Beijing (CN); Yuhua Shi, Beijing (CN); Qinggang Yin, Beijing (CN); Mengyue Wang, Beijing (CN); Yang Chu, Beijing (CN); Wenting Wang, Beijing (CN)

(21) Appl. No.: 19/048,825

(22) Filed: Feb. 7, 2025

(30) Foreign Application Priority Data
Feb. 9, 2024 (CN) 202410179060.8

(51) Int. Cl. C12N 9/10; C12P 19/18; C12P 19/44

(52) U.S. Cl. CPC C12N 9/1051; C12P 19/18; C12P 19/44

(57) ABSTRACT

The invention discloses a flavonoid glycoside glycosyltransferase LbUGT71BX1 in laportea bulbifera as well as the coding gene and the use thereof. The amino acid sequence of the flavonoid glycoside glycosyltransferase LbUGT71BX1 is shown as SEQ ID NO: 2. The nucleotide sequence of the coding gene of the flavonoid glycoside glycosyltransferase LbUGT71BX1 is shown as SEQ ID NO: 1. According to the invention, on the basis of relevant results of the second-generation transcriptome and the third-generation full-length transcriptome sequencing of the laportea bulbifera, the last-step key enzyme LbUGT71BX1 for the synthesis of the flavonoid glycoside in the laportea bulbifera is screened and identified by using a reverse genetics method, filling the terminal blank of the biosynthesis pathway of the flavonoid glycoside in the laportea bulbifera.

### CROSS-REFERENCE TO RELATED APPLICATION

This application claims priority of Chinese Patent Application No. 202410179060.8, filed on Feb. 9, 2024, the entire contents of which are incorporated herein by reference.

### REFERENCE TO A SEQUENCE LISTING SUBMITTED VIA EFS-WEB

The content of the xml file of the sequence listing named “P250205_sequence listing_ST26” which is 6813 bytes in size was created on Feb. 5, 2025 and electronically submitted via EFS_Web herewith. These sequence listing is incorporated herein by reference in its entirety.

### TECHNICAL FIELD

The invention relates to the field of biotechnology, in particular to flavonoid glycoside glycosyltransferase LbUGT71BX1 in laportea bulbifera, as well as the coding gene and the use thereof.

### BACKGROUND

Laportea bulbifera is a plant of the genus Laportea of the family Urticaceae, which is mainly native to Guizhou, etc. Its fresh or dried whole plant (also known as Hong He Ma in Chinese) is used by the local Miao and Buyi ethnic minorities for the treatment of rheumatic pain, numbness of limbs, traumatic injuries and other diseases. Runzao Zhiyang Capsule with Hong He Ma as the main raw material has good curative effect, ranking third in the field of dermatology and anorectal department in the scientific and technological competitiveness ranking of large varieties of traditional Chinese medicine in 2018. At present, the clinical application of laportea bulbifera mainly depends on the excavation of its wild resources. With the wide application of Hong He Ma medicinal material and its preparations, its wild resources are increasingly scarce, which seriously restricts the development of laportea bulbifera industry.

Flavonoids are widely distributed in plants, drugs and human diet. Modern studies have shown that flavonoids have anti-inflammatory, antioxidant and other biological activities, and play an important role in plant resistance to biotic and abiotic stresses. Laportea bulbifera grows in the karst area with high salinity and drought. Flavonoids and glycosides are one of the main chemical components in laportea bulbifera, but the study on its biosynthetic pathway and key enzymes is still in the blank.

UDP-glycosyltransferase (UGT) is involved in the glycosylation reaction of flavonoid glycoside biosynthesis pathway. At present, many UGT genes have been identified and reported to have flavonoid glycosyltransferase activity, such as MrUGT78R1 and MrUGT78W1 in Morella rubra, which can catalyze the synthesis of myricetin 3-O-rhamnose and 3-O-galactose, respectively. The 3-O-glucosylation of flavonoids can be catalyzed in Fragaria X ananassa. However, the number and functional diversity of UGTs in angiosperms are also changing due to the occurrence of tandem repeats, and further research on the function and catalytic mechanism of UGTs is still needed to lay the foundation for the metabolic engineering of flavonoids.

### SUMMARY

In view of this, the present invention provides the flavonoid glycoside glycosyltransferase LbUGT71BX1 in laportea bulbifera and the coding gene and the use thereof, and fills in the terminal blank of the flavonoid glycoside biosynthesis pathway in laportea bulbifera.

The technical scheme of the invention is as follows:

The amino acid sequence of the protein is shown as SEQ ID NO: 2.

The invention also provides a coding gene of the protein, and the nucleotide sequence of the coding gene is shown as SEQ ID NO: 1.

The gene is named LbUGT71BX1, and the protein encoded by it is named LbUGT71BX1. The specific information is as follows: the genetic sequence of the LbUGT71BX1 is shown as the sequence 1 in the sequence table, wherein the sequence 1 contains 1437 nucleotides which code the protein shown as the sequence 2 in the sequence table, and the sequence 2 consists of 478 amino acids.

The expression cassettes, recombinant expression vectors or recombinant bacteria containing the coding gene also belong to the protection scope of the invention.

The primer pair for amplifying the full length of the coding gene also belongs to the protection scope of the invention, and in the primer pair, one primer sequence is shown as SEQ ID NO: 3, and the other primer sequence is shown as SEQ ID NO: 4.

The use of the protein as glycosyltransferase also belongs to the protection scope of the invention.

The glycosyltransferase is an enzyme having any of the following functions:
(1) Catalyzing substrate kaempferol to generate kaempferol-3-O-galactoside when UDP-galactose is used as a sugar donor;
(2) Catalyzing substrate kaempferol to generate kaempferol-3-O-glucoside when UDP-glucose is used as a sugar donor;
(3) Catalyzing substrate myricetin is catalyzed to generate myricetin-3-O-galactoside when UDP-galactose is used as a sugar donor;
(4) Catalyzing substrate myricetin to generate myricetin-3-O-glucoside when UDP-glucose is used as a sugar donor;
(5) Catalyzing substrate gossypetin to generate gossypetin-3-O-glucoside when UDP-glucose is use as a sugar donor;
(6) Catalyzing substrate quercetagetin to generate quercetagetin-3-O-glucoside or quercetagetin-7-O-glucoside when UDP-glucose is use as a sugar donor;
The use of the protein in any of the following also belongs to the protection scope of the invention:

(1) Catalyzing substrate kaempferol to generate kaempferol-3-O-galactoside when UDP-galactose is used as a sugar donor;
(2) Catalyzing substrate kaempferol to generate kaempferol-3-O-glucoside when UDP-glucose is used as a sugar donor;
(3) Catalyzing substrate myricetin is catalyzed to generate myricetin-3-O-galactoside when UDP-galactose is used as a sugar donor;
(4) Catalyzing substrate myricetin to generate myricetin-3-O-glucoside when UDP-glucose is used as a sugar donor;
(5) Catalyzing substrate gossypetin to generate gossypetin-3-O-glucoside when UDP-glucose is use as a sugar donor;
(6) Catalyzing substrate quercetagetin to generate quercetagetin-3-O-glucoside or quercetagetin-7-O-glucoside when UDP-glucose is use as a sugar donor;
The use of the coding gene in any one of the following also belongs to the protection scope of the invention:

(1) Catalyzing substrate kaempferol to generate kaempferol-3-O-galactoside when UDP-galactose is used as a sugar donor;
(2) Catalyzing substrate kaempferol to generate kaempferol-3-O-glucoside when UDP-glucose is used as a sugar donor;
(3) Catalyzing substrate myricetin is catalyzed to generate myricetin-3-O-galactoside when UDP-galactose is used as a sugar donor;
(4) Catalyzing substrate myricetin to generate myricetin-3-O-glucoside when UDP-glucose is used as a sugar donor;
(5) Catalyzing substrate gossypetin to generate gossypetin-3-O-glucoside when UDP-glucose is use as a sugar donor;
(6) Catalyzing substrate quercetagetin to generate quercetagetin-3-O-glucoside or quercetagetin-7-O-glucoside when UDP-glucose is use as a sugar donor;

The invention is based on the related results of the second-generation transcriptome and the third-generation full-length transcriptome sequencing of the laportea bulbifera. The key enzyme LbUGT71BX1 of the last step in the synthesis of kaempferol-3-O-glucoside/galactoside, myricetin-3-O-glucoside/galactoside, myricetin-7-O-glucoside, gossypetin-3-O-glucoside and quercetagetin-3/7-O-glucoside is identified by a reverse genetics method, filling in the blank of the biosynthesis pathway of the flavonoid glycoside component in the laportea bulbifera and providing the glycosyltransferase protein and the coding sequence thereof for further biosynthesis of kaempferol-3-O-glucoside/galactoside, myricetin-3-O-glucoside/galactoside, myricetin-7-O-glucoside, gossypetin-3-O-glucoside and quercetagetin-3/7-O-glucoside.

The invention excavates and identifies the key UGT for biosynthesis of flavonoid glycoside active ingredients in the laportea bulbifera, has important significance for understanding the quality formation of the laportea bulbifera, and provides a key element for synthetic biology research of flavonoid ingredients.

### BRIEF DESCRIPTION OF DRAWINGS

For purposes of illustration and not limitation, the present invention will now be described in accordance with a preferred embodiment thereof, with particular reference to the accompanying drawings, in which:

FIG. 1 shows the molecular structural formula of kaempferol, myricetin, gossypetin, quercetagetin, kaempferol-3-O-glucoside/galactoside, myricetin-3-O-glucoside/galactoside, myricetin-7-O-glucoside, gossypetin-3-O-glucoside, and quercetagetin-3/7-O-glucoside.

FIG. 2 shows the expression of Lb UGT71BX1 gene in different tissues of laportea bulbifera. B: bulbil; F: flower; L: leaf, R: root; S: stem.

FIG. 3 shows the agarose gel electrophoresis diagram of LbUGT71BX1 gene cloning and vector construction. Lane 3 in the left figure shows the result of LbUGT71BX1 gene cloning, and FIGS. 8 to 14 on the right show the results of pMAL-c2X-LbUGT71BX1 vector construction. The remaining lanes are the results of other genes cloned in the same batch.

FIG. 4 shows the agarose gel electrophoresis diagram of the recombinant plasmid of the LbUGT71BX1 gene transferred into BL21 (DE3) Escherichia coli, wherein lanes 5 and 6 are LbUGT71BX1. The remaining lanes are the results of other genes transformed at the same time.

FIG. 5 shows the SDS-Page gel diagram of the recombinant purified protein LbUGT71BX1, wherein lane 3 is LbUGT71BX1. The remaining lanes are the results of other purified enzymes done at the same time.

FIG. 6a shows the extracted ion chromatogram of the sample tested by LC-Q-TOF-MS and the secondary mass spectrum fragment of the product P1 when the LbUGT71BX1 protein takes kaempferol as the substrate and UDP-galactose as the donor; FIG. 6 b shows the extracted ion chromatogram of the sample tested by LC-Q-TOF-MS and the secondary mass spectrum fragment of the product P2 when the LbUGT71BX1 protein takes kaempferol as the substrate and UDP-glucose as the donor.

FIG. 7a shows the extracted ion chromatogram of the sample tested by LC-Q-TOF-MS and the secondary mass spectrum fragment of the product P3 when the LbUGT71BX1 protein takes myricetin as the substrate and UDP-galactose as the donor; FIG. 7b shows the extracted ion chromatogram of the sample tested by LC-Q-TOF-MS and the secondary mass spectrum fragments of the products P4 and P5 when the LbUGT71BX1 protein takes myricetin as the substrate and UDP-glucose as the donor.

FIG. 8 shows the identification result of the catalytic product of recombinant protein LbUGT71BX1 on gossypetin identified by LC-Q-TOF-MS.

FIG. 9 shows the identification result of the catalytic product of recombinant protein LbUGT71BX1 on quercetagetin identified by LC-Q-TOF-MS.

### DETAILED DESCRIPTION

Hereinafter, the present invention will be described in detail with reference to the embodiments. The embodiments are for a better understanding of the invention, but are not intended to limit the invention. The experimental methods in the following implementation methods are all conventional methods, and the experimental reagents involved are all conventional biochemical reagents.

### Embodiment 1 Screening of UGTs Genes Based on the Third-Generation Full-Length Transcriptome and Second-Generation Transcriptome Data of Laportea bulbifera

### 1.1 Experimental Method

The non-patent document of laportea bulbifera (describing laportea bulbifera): Wang, W., Wang, X., Shi, Y et al. Identification of Laportea bulbifera using the complete chloroplast genome as a potentially effective super-barcode. J Appl Genetics 64, 231-245 (2023). https://doi.org/10.1007/s13353-022-00746-4). The laportea bulbifera was collected from Leigong Mountain in the southeast of Guizhou Province, and divided into five parts: root, stem, leaf, flower and bulbil. The second and third generations were sequenced by Illumina platform and PacBio platform.

The UGT protein sequence of Arabidopsis thaliana was downloaded from Arabidopsis thaliana database (https://www.arabidopsis.org/), and Blast homology comparison analysis was carried out between the UGT protein sequence of Arabidopsis thaliana and the full-length transcriptome database of laportea bulbifera. A hidden Markov model (HMM) file of the UGT protein was downloaded from Pfam (http://pfam-legacy.xfam.org/), and the protein sequences containing the UDPGT. HMM (PF00201) domain were searched using HMMER (http://hmmer.janelia.org/static/binaries/hmmer3.0) software. The results of Blast homology analysis and HMMER domain search were combined to screen out the sequences with similarity less than 30%, e value less than 10-5 and length less than 300 amino acids. The above transcripts were submitted to NCBI-CDD (https://www.ncbi.nlm.nih.gov/Structure/bwrpsb/bwrpsb.cgi) for validation of the conserved domain of UGT, and the results were visualized using TBtools software. Protein sequences without UGT conserved domains were screened out. A conservative domain search was performed on the above transcripts using the Motif website (https://meme-suite.org/meme/), and only protein sequences with PSPG-box specific domains were retained. The protein sequence obtained by screening in the above steps was identified as the UGT gene family member (LbUGTs) of laportea bulbifera.

Using the third generation full-length transcript as a reference, the Clean Reads obtained from the second generation transcriptome were aligned with the third generation transcript by HiSAT2 to obtain the position information of the transcript. The expression levels of transcripts in different tissues were quantitatively analyzed by RSEM software, and the FP-KM values were calculated. DESeq2 software was used for differential expression analysis to further screen out LbUGTs genes with higher expression level.

### 1.2 Results and Analysis

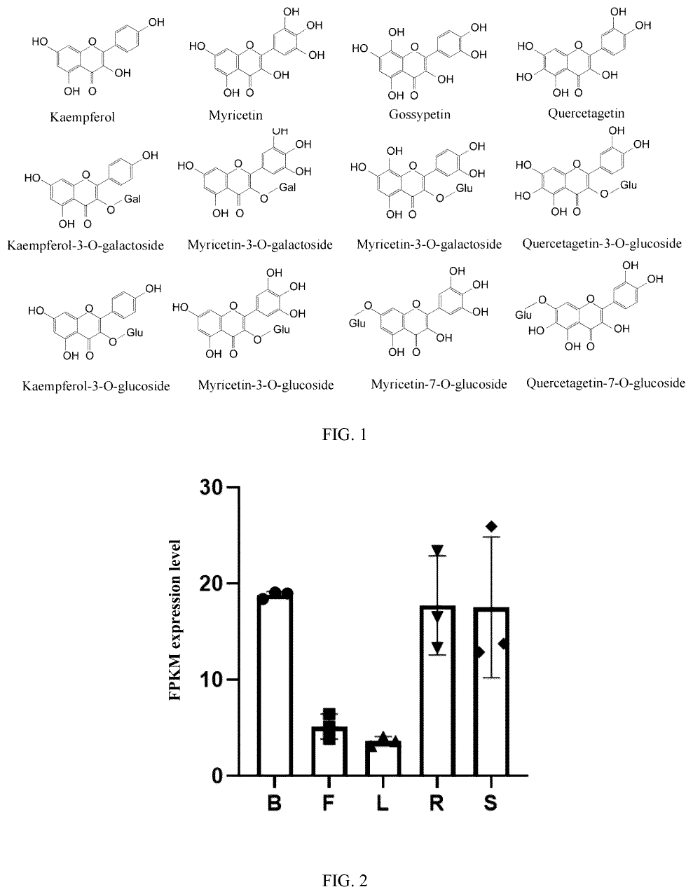

Based on the full-length transcriptome data of the third generation, 114 UGTs with PSPG-box conserved domains were screened. Based on the differential expression analysis of the second generation transcriptome, it was found that LbUGT71BX1 was highly expressed in the bulbils, roots and stems of the bulbils (FIG. 2), and it was speculated that it might have glycosyltransferase activity.

### Embodiment 2 Cloning of Candidate LbUGT71BX1 Gene in Laportea bulbifera and Construction of Expression Vector

### 2.1 Experimental Method

The pMAL-c2X-LbUGT71BX1 expression vector was obtained by homologous recombination to design the primer sequence (as shown in Table 1). The LbUGT71BX1 gene fragment was cloned using the KOD high-fidelity enzyme (KOD high-fidelity enzyme PCR system in a total volume of 50 μL:25 μL KOD One™ PCR Master Mix, 1.5 μL primer (10 mM), 1 μL template, and 21 μL water, as programmed in Table 2).

TABLE 1
Primer Sequences Used for Cloning the LbUGT71BX1 Gene

| Gene number | Primer name | Primer sequence |
|---|---|---|
| LbUGT71BX1 | LbUGT71BX1F | AGGATTTCAGAATTCGGATCCATGAATTCA |
| | | TTGATAGAGCTTATCTTCG |
| | LbUGT71BX1R | CAAGCTTGCCTGCAGGTCGACTCAAGCCA |
| | | CAACCTTAGAAACGT |

TABLE 2
KOD High Fidelity Enzyme PCR Reaction Procedure

| Step | Temperature (° C.) | Time (s) | Number of cycles |
|---|---|---|---|
| Step 1 | 95 | 180 | 1 |
| Step2 | 98 | 10 | 40 |
| | 57 | 10 | |
| | 68 | 20 | |
| Step3 | 68 | 300 | 1 |

The target gene was constructed into pMAL-c2X vector (purchased from New England Biolabs. With product catalog number of E8200S) using ClonExpress II One Step Cloning Kit (purchased from Nanjing Vazyme Biotechnology Co., Ltd., catalogue number C112), and the restriction sites were BamHI and SalI. According to the instruction, the optimal molar ratio of the amount of the cloning vector to the amount of the insert used in the reaction system was 1:2, and the optimal amount was calculated by using the following formula:

Optimum amount of cloning vector used=[0.02×number of base pairs of cloning vector] ng (0.03 μmol)

Optimum amount of insert=[0.04×number of base pairs of insert] ng (0.06 μmol)

Through calculation, the composition of the reaction system is shown in Table 3:

TABLE 3
Composition of Homologous Recombination Reaction System

| Component | Sample volume | |
|---|---|---|
| Linearized pMAL-c2X vector | 4.0 μL (about 134 ng) | |
| Clone fragment | 1.0 μL (about 60 ng) | |
| 5 × CE II Buffer | 4.0 | μL |
| Exnase II | 2.0 | μL |
| ddH₂O | 39.0 | μL |
| Total | 50.0 | μL |

After the addition of all the components, the reaction solution was gently pipetted for mixing, followed by instantaneous centrifugation, and incubated at 37° C. for 30 minutes to obtain the pMAL-c2X-LbUGT71BX1 recombinant product.

The transformation steps of the recombinant product are as follows: (1) Thaw Trans1-T1 competent cells (purchased from Beijing TransGen Biotech Co., Ltd., catalogue number CD501-02) on ice. Aliquote 50 μL of thawed Trans1-T1 competent cells into precooled 1.5 mL centrifuge tubes. (2) Add 5 μL of the reconstituted product to the centrifuge tube, mix gently, and ice bath for 30 minutes. (3) After heat shock in a water bath at 42° C. for 30 seconds, transfer the centrifuge tube quickly to an ice bath for 2 minutes without shaking. (4) Pipette 50 μL of the transformed competent cells, spread them evenly on the LB agar medium with ampicillin resistance, and invert the plate and culture in the incubator at 37° C. for 12-16 hours.

The screening procedure for positive clones is as follows: (1) Select seven clones grown on LbUGT recombinant plates and incubate in 200 μL LB liquid medium (ampicillin resistance) for 2 hours at 37° C. (200 rpm). (2) Perform colony PCR identification using 2×Taq Mix DNApolymerase (purchased from Nanjing Vazyme Biotechnology Co., Ltd., catalogue number P131-01). The amplification primer sequence designed by Primer 6.0 is: pMAL-c2X-F: GTCGTCAGACTGTCGATGAAG; pMAL-c2X-R: GATGTGCTGCAAGGCGATT. The conventional PCR reaction system is shown in Table 4. The routine PCR reaction procedure is shown in Table 5. (3) Take 5 μL of PCR products for agarose gel electrophoresis testing, refer to the position of DNA Marker (purchased from Beijing TransGen Biotech Co., Ltd., catalogue number BM111-01), select the bacterial fluid with reasonable band position and send it to Beijing SinoGenoMax Research Center Co., Ltd. for testing. Then, align with the RNAseq sequence to obtain the final sequence information.

TABLE 4
Composition of Conventional PCR Reaction System

| Component | Sample volume | |
|---|---|---|
| 2 × Green Taq Mix | 10 | μL |
| 10 μM Primer F/R | 2 × 1 | μL |
| Bacterial fluid | 1.0 | μL |
| ddH₂O | 7.0 | μL |
| Total | 20.0 | μL |

TABLE 5
Routine PCR Reaction Procedure

| Temperature | Time | |
|---|---|---|
| 95° C. | Pre-denaturation for 3 min | |
| 95° C. | Denaturation for 15 s | 35 cycles |
| 60° C. | Annealing for 15 s | |
| 72° C. | Extend for 2 min | |
| 72° C. | Extend completely for 5 min | |

### 2.2 Experimental Results

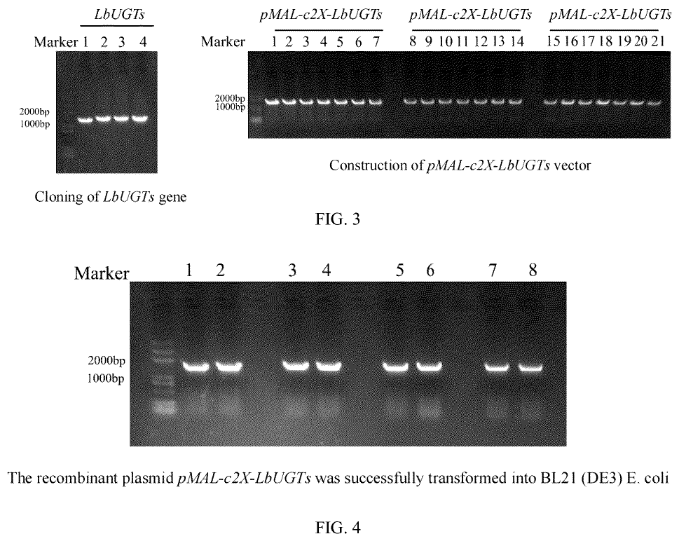

LbUGT71BX1 was obtained by cloning the primers in Table 1, and the results of agarose gel electrophoresis are shown in FIG. 3. After sequencing, the nucleotide sequence was found to be 99% similar to the original data by Blast, which was based on the actual sequencing results. The actual sequencing result is shown in the sequence 1, which contains 1437 nucleotides, and the protein shown in the sequence 2 in the coding sequence table consists of 478 amino acids. The gene is named LbUGT71BX1, and the protein encoded by it is named LbUGT71BX1.

### Embodiment 3 Functional Validation of Candidate LbUGT71BX1 Gene

### 3.1 Experimental Method

The transformation steps of the recombinant plasmid are as follows: (1) thaw BL21 (DE3) competent cells (purchased from Tiangen Biotech (Beijing) Co., Ltd., catalogue number CB105-02) on ice. Aliquote 50 μL of thawed BL21 (DE3) competent cells into precooled 1.5 mL centrifuge tubes. (2) Add 5 μL of pMAL-c2X-LbUGT71BX1 recombinant plasmid into a centrifuge tube, gently mix, and ice bath for 30 minutes. (3) After heat shock in a water bath at 42° C. for 90 seconds, transfer the centrifuge tube quickly to an ice bath for 2 minutes without shaking. (4) Pipette 50 μL of the transformed competent cells, spread them evenly on the LB agar medium with ampicillin resistance, and invert the plate and culture in the incubator at 37° C. for 12-16 hours.

The steps of inducing expression are as follows: (1) Select the monoclone on the transformed LB solid culture medium, and perform colony conventional PCR validation, wherein the method is the same as the above. (2) Pipette 80 μL of positive clones into 20 mL of LB liquid medium (ampicillin resistance) for overnight culture (37° C., 200 rpm). (3) Pipette 2 mL of the bacterial fluid cultured overnight, transfer it to 40 mL of LB liquid medium (ampicillin resistance), and culture at 37° C. and 200 rpm until the OD600 of the bacterial fluid is 0.8 (about 2 hours). (4) Add 15 μL of IPTG (1 M, isopropyl-β-D-thiogalactopyranoside) into the bacterial fluid, shake well, and induce the culture at 16° C. and 110 rpm for 16-24 hours.

The protein purification steps are as follows: collect the induced bacterial fluid, centrifuge at 7800 rpm at 4° C. for 5 minutes, and discard the supernatant; wash the bacterial precipitate with 3 mL of precooled Tris-HCl (pH 7.5), centrifuge at 7800 rpm at 4° C. for 3 minutes, and discard the supernatant. Add 1.5 mL Tris-HCl (pH 7.5) buffer to resuspend the bacterial cells and transfer to 2 mL centrifuge tube. Use an ultrasonic cell disruptor (30 Hz, working for 5 seconds and stopping for 5 seconds) to disrupt the resuspended bacterial fluid for 20 minutes. The color will change from milky white to translucent. The whole process is carried out on ice. Centrifuge the resuspended bacterial fluid at 12000 rpm for 30 minutes at 4° C., and the supernatant is the crude enzyme solution.

The instructions of PurKine™ Maltose Binding Protein Tag Protein Purification Kit (purchased from Abbkine Scientific Co., Ltd., catalogue number KTP2020) were referred to for the protein purification process. The whole process was carried out in a refrigerator at 4° C. The specific process is as follows: (1) Fix the gravity column, remove the plugs at both ends, and drain the protective solution; (2) Add 2 mL of binding buffer to the column to balance the resin, drain and repeat 3 times; (3) Mix the crude enzyme solution with the same volume of binding buffer to prepare the protein sample. Add the protein sample to the column for incubation, and collect the flow-through solution and repeat for 5 times; (4) add 2 mL of washing solution to the column to remove non-specific proteins, and repeat for 6 times, and test the protein content with Coomassie brilliant blue solution during the process. (5) Add 2 mL of elution buffer to the column to elute the specific protein (containing maltose tag), repeat 5 times, and test the protein content with Coomassie brilliant blue solution during the process. The eluent collected in this step is the purified protein solution. (6) Desalination and concentration treatment: add the purified protein solution to an ultrafiltration tube (30 kDa), centrifuge at 3800×g for 30 minutes at 4° C., and add 10 mL of protein replacement solution (50 mM Tris-HCl (pH 7.5), 10 mM DTT) to the tube in batches, and centrifuge again. The remaining solution in the suction tube is the desalted purified enzyme solution. (7) Use the Easy Protein Quantitative Kit (Bradford) protein concentration determination kit (purchased from Beijing TransGen Biotech Co., Ltd., catalogue number DQ101-01) to establish the protein concentration standard curve, and determine the protein concentration according to its instructions. (8) SDS-Page protein gel testing: take 80 μL of purified enzyme solution, add 20 μL of 5×protein loading buffer, mix well, boil for 5 minutes, and centrifuge at 12000 rpm for 5 minutes. Take 40 μL of the supernatant for electrophoresis at 150 V for 40-50 minutes until the bromophenol blue indicator band reaches the bottom of the silica gel, and then end the electrophoresis. After staining for 30 minutes, place it in the destaining solution for 1 hour, and then change the destaining solution for overnight destaining. On the next day, place the protein gel in the scanner for imaging.

The in-vitro enzyme activity testing steps are as follows: the 50 μL reaction system contained 1 μL flavonoid substrate (40 mM) (kaempferol or myricetin or gossypetin or quercetagetin), 1 μL UDP-sugar donor (100 mM) (UDP-galactose or UDP-glucose) and 10 g purified enzyme, and Tris-HCl buffer (pH 7.5) was used to complement the system. With the pMAL-c2X empty crude enzyme catalytic system as a negative control, place the reaction system at 37° C. for 1 hour, and add the same volume of methanol to stop the reaction. Concentrate the above mixture in vacuo and reconstitute it with 100 μL of methanol. Perform filtration using a 0.22 m microporous membrane and transfer the sample to a liquid vial for assay. Use the Agilent 1290 Infinity II-6430 Q-TOF to test the sample. The specific liquid conditions are as follows: (1) Column: ACQUITY UPLC BEH C18 1.7 m 2.1×100 mm; (2) Mobile phase: 0.1% pure water for phase A and chromatographic acetonitrile for phase B; (3) Flow rate: 0.3 mL/min; (4) Column temperature: 40° C.; (5) Injection volume: 1.0 μL; (6) Elution conditions are shown in Table 6. The mass spectrometry conditions are: the ion mode is negative ion, the scan range is 100-3000; the mode is Automated MS/MS; the ion fragment collision energy is 20 V; the source temperature is 350° C.; the nebulizer pressure is set to 30 psi.

TABLE 6
Mobile Phase Elution Gradient during LC-Q-TOF Test

| Time (min) | Phase A | Phase B |
|---|---|---|
| 0.0 | 95% | 5% |
| 2.0 | 95% | 5% |
| 10.0 | 40% | 60% |
| 10.5 | 5% | 95% |
| 12.5 | 5% | 95% |
| 13.0 | 95% | 5% |
| 16.0 | 95% | 5% |

### 3.2 Experimental Results

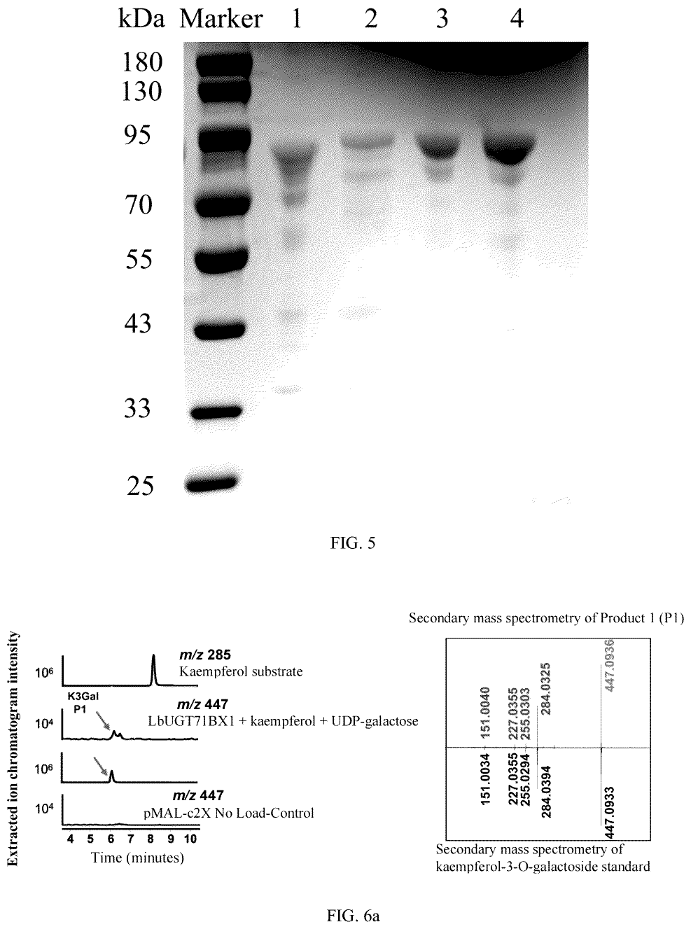

The pMAL-c2X-LbUGT71BX1 recombinant protein was successfully transformed into Escherichia coli (FIG. 4), and the recombinant protein was expressed (FIG. 5), and its function was identified by further enzyme activity analysis. The donor of enzyme activity reaction was UDP-glucose or UDP-galactose, and the acceptor was kaempferol (C₁₅H₁₀O₆, purchased from Shanghai Yuanye Biotechnology Co., Ltd., catalogue number: B21126) or myricetin (C15H10O8, purchased from Shanghai Yuanye Bio-Technology Co., Ltd., catalogue number B21458) or gossypetin (C15H1008,purchased from Shanghai Yuanye Bio-Technology Co., Ltd., catalogue number B29179) or quercetagetin (C₁₅H₁₀O₈, purchased from Shanghai Yuanye Bio-Technology Co., Ltd., catalogue number B29299). LbUGT71BX1 was identified by mass spectrometry, and it was found that there were product peaks (P1-P8) for the enzyme activities of the receptors kaempferol, myricetin, gossypetin and quercetagetin.

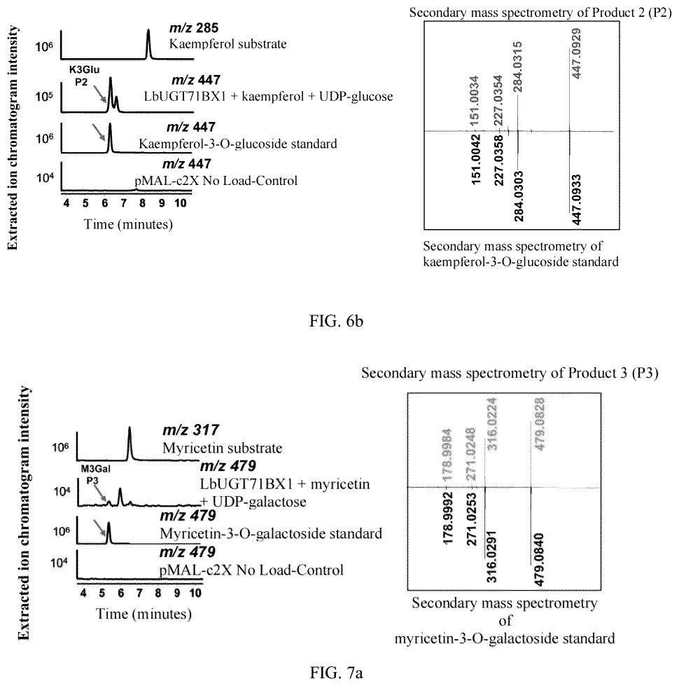

Compared with the test result of a pMAL-c2X-containing no-load crude enzyme catalytic system, when the kaempferol was used as the substrate and the UDP-galactose was used as the sugar donor, the product P1 had the same retention time and mass spectrometry fragmentation pattern as kaempferol-3-O-galactoside (K3Gal) ([M-H]—: m/z=447.0935, [M-H-Gal]-: m/z=284.0325)(FIG. 6a); when kaempferol was used as the substrate and UDP-glucose was used as the sugar donor, the product P2 had the same retention time and mass spectrometry fragmentation pattern as kaempferol-3-O-glucoside (K3Glu) ([M-H]—: m/z=447.0920, [M-H-Glu]—: m/z=284.0315) (FIG. 6b).

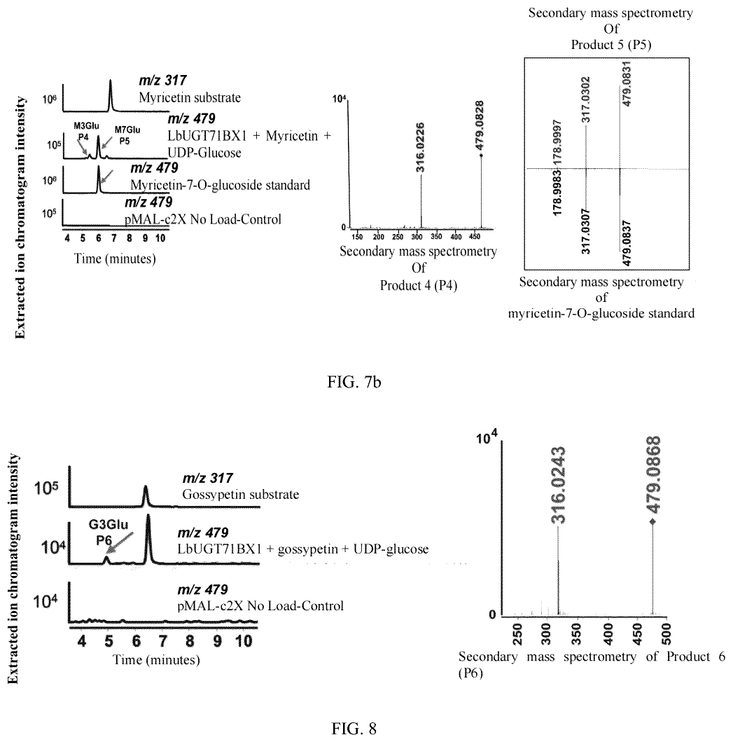

Compared with the test result of a pMAL-c2X-containing no-load crude enzyme catalytic system, when myricetin was used as the substrate and UDP-galactose was used as the sugar donor, the product P3 had the same retention time and mass spectrometry fragmentation pattern as myricetin-3-O-galactoside (M3Gal) ([M-H]—: m/z=479.0830, [M-H-Gal]—: m/z=316.0224)(FIG. 7a); when myricetin was used as the substrate and UDP-glucose was used as the sugar donor, the product P4 was identified as myricetin-3-O-glucoside (M3Glu) ([M-H]—: m/z=479.0828, [M-H-Glu]—: m/z=316.0226) according to the fragmentation pattern, and the product P5 had the same retention time and mass spectrometry fragmentation pattern as myricetin-7-O-glucoside (M7Glu) ([M-H]—: m/z=479.0832, [M-H-Glu]—: m/z=317.0303) (FIG. 7b).

Compared with the test result of the pMAL-c2X-containing no-load crude enzyme catalytic system, when the gossypetin was used as the substrate and the UDP-glucose was used as the sugar donor, the product P6 was identified as gossypetin-3-O-glucoside (G3Glu) ([M-H]—: m/z=479.0868, [M-H-Glu]—: m/z=316.0243) according to the fragmentation pattern (FIG. 8).

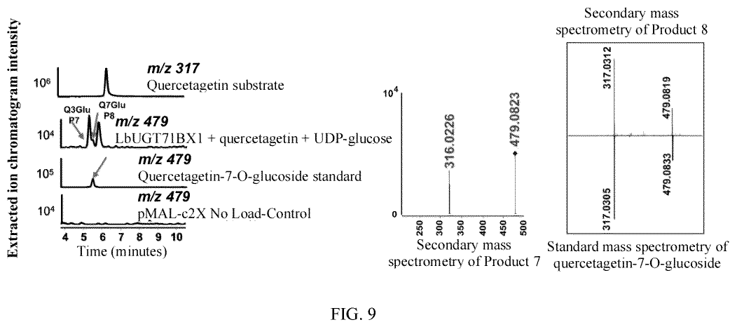

Compared with the test result of the pMAL-c2X-containing no-load crude enzyme catalytic system, when quercetagetin was used as the substrate and UDP-glucose was used as the sugar donor, the product P7 was identified as quercetagetin-3-O-glucoside (Q3Glu) ([M-H]—: m/z=479.0823, [M-H-Glu]—: m/z=316.0226), and the product P8 had the same retention time and mass spectrometry fragmentation patter as quercetagetin-7-O-glucoside (Q7Glu) ([M-H]—: m/z=479.0797, [M-H-Glu]—: m/z=317.0305) (FIG. 9).

To sum up, based on the mass spectrometry test data, the glycosyltransferase LbUGT71BX1 can catalyze kaempferol to generate kaempferol-3-O-glucoside/galactoside, catalyze myricetin to generate myricetin-3-O-glucoside/galactoside and myricetin-7-O-glucoside, catalyze gossypetin to generate gossypetin-3-O-glucoside, and catalyze quercetagetin to generate quercetagetin-3/7-O-glucoside.

In the invention, the sequence of the flavonoid glycosyltransferase in the laportea bulbifera is obtained mainly through third-generation full-length transcriptome analysis, and when UDP-glucose is used as donor through prokaryotic expression the recombinant protein LbUGT71BX1 can catalyze kaempferol to generate kaempferol-3-O-glucoside, catalyze myricetin to generate myricetin-3/7-O-glucoside, catalyze gossypetin to generate gossypetin-3-O-glucoside, and catalyze quercetagetin to generate quercetagetin-3/7-O-glucoside; when UDP-galactose is used as donor, it can catalyze kaempferol to generate kaempferol-3-O-galactoside and catalyze myricetin to generate myricetin-3-O-galactoside.

The above specific embodiments do not limit the scope of the present invention. Those skilled in the art understand that various modifications, combinations, subcombinations, and substitutions may occur, depending upon design requirements and other factors. Any modification, equivalent substitution and improvement within the spirit and principle of the invention should be included in the protection scope of the invention.

1. A protein, the amino acid sequence of which is shown in SEQ ID NO: 2.

2. The coding gene of the protein of claim 1, wherein the nucleotide sequence of the coding gene is shown as SEQ ID NO: 1.

3. The expression cassette, recombinant expression vector or recombinant bacterium containing the coding gene of claim 2.

4. The primer pair for amplifying the full length of the coding gene of claim 2, wherein one primer sequence is shown in SEQ ID NO: 3, and the other primer sequence is shown in SEQ ID NO: 4.

5. The use of the protein of claim 1 as a glycosyltransferase.

6. The use according to claim 5, wherein the glycosyltransferase is an enzyme having any of the following functions:
(1) Catalyzing substrate kaempferol to generate kaempferol-3-O-galactoside when UDP-galactose is used as a sugar donor;
(2) Catalyzing substrate kaempferol to generate kaempferol-3-O-glucoside when UDP-glucose is used as a sugar donor;
(3) Catalyzing substrate myricetin is catalyzed to generate myricetin-3-O-galactoside when UDP-galactose is used as a sugar donor;
(4) Catalyzing substrate myricetin to generate myricetin-3-O-glucoside when UDP-glucose is used as a sugar donor;
(5) Catalyzing substrate gossypetin to generate gossypetin-3-O-glucoside when UDP-glucose is use as a sugar donor;
(6) Catalyzing substrate quercetagetin to generate quercetagetin-3-O-glucoside or quercetagetin-7-O-glucoside when UDP-glucose is use as a sugar donor;

7. The use of the protein of claim 1 in any of the following:
(1) Catalyzing substrate kaempferol to generate kaempferol-3-O-galactoside when UDP-galactose is used as a sugar donor;
(2) Catalyzing substrate kaempferol to generate kaempferol-3-O-glucoside when UDP-glucose is used as a sugar donor;
(3) Catalyzing substrate myricetin is catalyzed to generate myricetin-3-O-galactoside when UDP-galactose is used as a sugar donor;
(4) Catalyzing substrate myricetin to generate myricetin-3-O-glucoside when UDP-glucose is used as a sugar donor;
(5) Catalyzing substrate gossypetin to generate gossypetin-3-O-glucoside when UDP-glucose is use as a sugar donor;
(6) Catalyzing substrate quercetagetin to generate quercetagetin-3-O-glucoside or quercetagetin-7-O-glucoside when UDP-glucose is use as a sugar donor;

8. The use of the coding gene of claim 2 in any of the following:
(1) Catalyzing substrate kaempferol to generate kaempferol-3-O-galactoside when UDP-galactose is used as a sugar donor;
(2) Catalyzing substrate kaempferol to generate kaempferol-3-O-glucoside when UDP-glucose is used as a sugar donor;
(3) Catalyzing substrate myricetin is catalyzed to generate myricetin-3-O-galactoside when UDP-galactose is used as a sugar donor;
(4) Catalyzing substrate myricetin to generate myricetin-3-O-glucoside when UDP-glucose is used as a sugar donor;
(5) Catalyzing substrate gossypetin to generate gossypetin-3-O-glucoside when UDP-glucose is use as a sugar donor;
(6) Catalyzing substrate quercetagetin to generate quercetagetin-3-O-glucoside or quercetagetin-7-O-glucoside when UDP-glucose is use as a sugar donor;


SEQUENCE LISTING (4 sequences)

SEQ ID NO: 1 - DNA, 1437 bp, Laportea bulbifera
  source 1..1437: mol_type = other DNA
atgaattcattgatagagcttatcttcgtcccggctcttggggccggccacttggtctcggccgtggagatggcaaagctccttgtggcccgagactctcacctctccgtctccgttctccttctcaagcacccggtggaatcggtaagcaatgtcagcgacgagtctctctcttcttttgagcgcatcaagctcgtcgatctcccgcaggacgagcccgccgccgactccactcagctcgccttcttacgcactttctaccagagccagaaaccccacatccgcaacgccgtcagcaggattattgcgtcctgtggctcgacccggctggccgggttcgtgttggacatgctttgtacgccgatgattgacgtggccgacgagtttggagtcccttcatacgtctacttcacgtccggcgccgggtcgatttgcctgctgaatcaactgctcagcggccatgaggagagcgacaacttcgccgagtttaaggaggaggtcgtcgtgccggggttcgccaacccggttccagagaaggccttgccttctatcctactggataaggccatggctccgtggctttacggcatctgcagagggatgtcaaaggccaagggcgttttggtcaacacgtttgcggagcttgaagcgagcatgatccacgctctgtcgacatccaacaagttccctcctgtttatccagtggggcccataattgatctcggcccccgggacagcagccacgggaggaaggcggagatcgtggagtggctggaccggcagcctccttcctcggtggtgttcctgtgcttcgggagcatggagagcttcggcggcaatcaggtgagagagatcgcggccgcgctggagcgcagcggggttcggttcctgtggtccctacgccgaccctcgcagaagcacaaggtccggacgccgaccgactacgaggatccgtcggaagtgctgccggaggggttcatggagcggacggccgcggtggggaaggtcgtcgggtgggccccgcagacggtggtgctgggccacccggcggtgggagggttcgtgtcgcactgcgggtggaactccatcctggagagtgtgtggttcggagtgccggtggcctcatggccggtgaacgcggagcagcatctaaacgcgttagagatggtaaatgagttgggattggcggtggagatcaccctggactaccggaaggatttcaaaaaggagaggtcggtggagattgataatgggaatatgattgtgaaggcagacactatagagagaggaatacataagttgatggagtatgactgtgacacaaggaagagggtaaaggagatgagagagaaaagcagagatgccttgcttcacggaggctcctcctacacttcactgtcccatttcattactgacgtttctaaggttgtggcttga

SEQ ID NO: 2 - protein, 478 aa, Laportea bulbifera
MNSLIELIFVPALGAGHLVSAVEMAKLLVARDSHLSVSVLLLKHPVESVSNVSDESLSSFERIKLVDLPQDEPAADSTQLAFLRTFYQSQKPHIRNAVSRIIASCGSTRLAGFVLDMLCTPMIDVADEFGVPSYVYFTSGAGSICLLNQLLSGHEESDNFAEFKEEVVVPGFANPVPEKALPSILLDKAMAPWLYGICRGMSKAKGVLVNTFAELEASMIHALSTSNKFPPVYPVGPIIDLGPRDSSHGRKAEIVEWLDRQPPSSVVFLCFGSMESFGGNQVREIAAALERSGVRFLWSLRRPSQKHKVRTPTDYEDPSEVLPEGFMERTAAVGKVVGWAPQTVVLGHPAVGGFVSHCGWNSILESVWFGVPVASWPVNAEQHLNALEMVNELGLAVEITLDYRKDFKKERSVEIDNGNMIVKADTIERGIHKLMEYDCDTRKRVKEMREKSRDALLHGGSSYTSLSHFITDVSKVVA

SEQ ID NO: 3 - DNA, 49 bp, synthetic construct
  source 1..49: mol_type = other DNA
aggatttcagaattcggatccatgaattcattgatagagcttatcttcg

SEQ ID NO: 4 - DNA, 44 bp, synthetic construct
  source 1..44: mol_type = other DNA
caagcttgcctgcaggtcgactcaagccacaaccttagaaacgt